(19) United States
(12) Patent Application Publication
(10) Pub. No.: US 2023/0307122 A1
(43) Pub. Date: Sep. 28, 2023

(54) CLOUD LIFE MONITORING SYSTEM

(71) Applicant: Wen-Hsiung Liu, Kaohsiung City (TW)

(72) Inventor: Wen-Hsiung Liu, Kaohsiung City (TW)

(73) Assignee: Wen-Hsiung Liu, Kaohsiung City (TW)

(21) Appl. No.: 17/932,279

(22) Filed: Sep. 14, 2022

(30) Foreign Application Priority Data
Mar. 22, 2022 (TW) 111202813

(51) Int. Cl. G16H 40/67; A61B 5/00; A61B 5/0205

(52) U.S. Cl. CPC G16H 40/67; A61B 5/002; A61B 5/0022; A61B 5/0205

(57) ABSTRACT

The invention is related to a cloud life monitoring system for timely monitoring data of blood oxygen and/or heart beat of a to-be-tested human, comprising a cloud computer for executing software for cloud monitoring, including: an input module for imputing data of blood oxygen and/or heart beat of the to-be-tested human timely; a processing module for receiving the data of the blood oxygen and/or heart beat and processing; a storing module for storing safety data record of the blood oxygen and/or heart beat, wherein the processing module further includes a comparison sub-module for comparing the timely data and for judging if the data is deviating from the corresponding safety data; and a notification module for simultaneously notifying the related people and the medicare institution.

### CROSS-REFERENCE TO RELATED APPLICATIONS

This application claims priority to Taiwan Application No. 111202813 filed on Mar. 22, 2022, the entire content of which is incorporated herein by reference.

### FIELD OF THE INVENTION

The invention is related to a cloud life monitoring system to timely and continuously receive the blood oxygen and/or heart beat data of a to-be-tested human wearing a blood oxygen device and transfer the data to a cloud computer through a wireless network via a mobile phone, and to timely inform the related people and medicare institution upon determining the to-be-tested human being subject to a serious abnormal condition, thereby accomplishing an emergency aid task in a complete process.

### BACKGROUND OF THE INVENTION

At present, the new crown pneumonia virus has still been in spreading. On the other hand, sudden occurrence of a lot of patients with heart beat or blood oxygen problem results in worrying about sudden death due to hypoxia, while the hospitals are corrupted due to our-of-order medical management resulting from entering of a huge patients.

A resolution thereto is to wear a blood oxygen device, providing with the function of detecting the blood oxygen and/or heart beat and transferring the data of the blood oxygen and/or heart beat to a mobile phone via a Bluetooth App. However, such an approach is merely for static reading without providing timely rescuing during emergency, and thus affecting life safety. Therefore, it would be an expected emergency aid in the medical field to develop a mechanism in which a cloud computer is utilized to continuously receive the data of the blood oxygen and/or heart beat in real time of 24 hours and a LINE (trademark) notification mechanism is designed such that under the condition that the real-time data is exceeding a safety range, the user, i.e. the patient, and/or the taking-care people, will be notified via the mobile phone and thereafter the medicare institution will be notified for providing first-aid in real time.

### SUMMARY OF THE INVENTION

An objective of the invention is to provide a cloud life monitoring system, providing with a cloud computer for processing data of the blood oxygen and/or heart beat continuously detected by and transferred from a blood oxygen device in real time and notifying the related people and the medicare institution when the patient is in a serious abnormal condition, in which the life of the patient will be largely secured based on the accuracy of the processing of the cloud computer.

A further objective of the invention is to provide a cloud life monitoring system to notify via a mobile phone as the medicae staff is not staying beside the patient's bed just when the patient is in a serious abnormal condition, thereby reducing the nerving and stress of the medicare staff.

Another objective of the invention is to provide a cloud computer utilizing a cloud detecting system, which is cheaper in cost and capable of detecting the patients in a large scale so as to constitute a cloud ICU, rendering a family or a clinic using fewer budget to build a simple ICU.

A still further objective of the invention is to provide a cloud computer for receiving the data of the blood oxygen and/or heart beat in real time and connecting with a LINE network to inform via the mobile phone the taking-care people when the real time data in the cloud computer exhibits an abnormal condition.

The present invention is to provide a cloud life monitoring system for timely monitoring data of blood oxygen and/or heart beat of a to-be-tested human, comprising a cloud computer for executing software for cloud monitoring, including:
an input module for imputing data of blood oxygen and/or heart beat of the to-be-tested human timely, and continuously transferring the data from a mobile phone via a wireless network;
a processing module, coupled to the input module for receiving the data of the blood oxygen and/or heart beat and processing;
a storing module with the processing module, for storing safety data record of the blood oxygen and/or heart beat, historic record data of the blood oxygen and/or heart beat of the to-be-tested human, and the timely data of the blood oxygen and/or heart beat of the to-be-tested human, and for storing ID authentication data of related people and pre-set data of a medicare institution, in which the related people is the to-be-tested human or a people fro taking care, wherein the processing module further includes a comparison sub-module for comparing the timely data of the blood oxygen and/or heart beat of the to-be-tested human with its corresponding safety data, and for judging if the data of the blood oxygen and/or heart beat of the to-be-tested human is deviating from the corresponding safety data, rendering the to-be-tested human to be in a serious abnormal condition; and
a notification module, coupled to the processing module for simultaneously notifying the related people and the medicare institution via the wireless network or the mobile phone by transmitting a notification signal when it is determined that it is in a serious abnormal condition due to deviation from the safety data after comparison,
whereby, the cloud life monitoring system is to timely monitor the data of the blood oxygen and/or heart beat of the to-be-tested human and to provide timely rescuing in emergency, so as to build a simple and efficient ICU system.

### BRIEF DESCRIPTION OF THE DRAWINGS

FIG. 1 shows a structural block diagram of a cloud computer according to the invention.

### DETAILED DESCRIPTION OF THE INVENTION

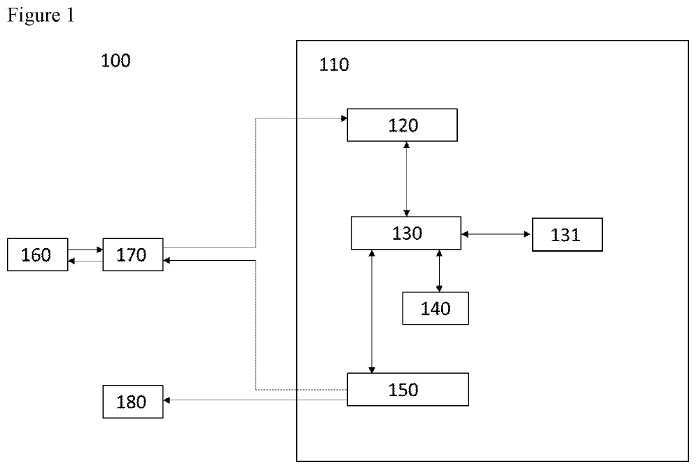

As shown in the block diagram of FIG. 1, it is provided in the invention a cloud life monitoring system 100 for timely monitoring data of blood oxygen and/or heart beat of a to-be-tested human, comprising a cloud computer 110 for executing software for cloud monitoring, including:
an input module 120 for imputing data of blood oxygen and/or heart beat of the to-be-tested human timely, and continuously transferring the data from a mobile phone 160 via a wireless network 170;
a processing module 130, coupled to the input module 120 for receiving the data of the blood oxygen and/or heart beat and processing;
a storing module 140, coupled with the processing module 130, for storing safety data record of the blood oxygen and/or heart beat, historic record data of the blood oxygen and/or heart beat of the to-be-tested human, and the timely data of the blood oxygen and/or heart beat of the to-be-tested human, and for storing ID authentication data of related people and pre-set data of a medicare institution 180, in which the related people is the to-be-tested human or a people for taking care,
wherein the processing module 130 further includes a comparison sub-module 131 for comparing the timely data of the blood oxygen and/or heart beat of the to-be-tested human with its corresponding safety data, and for judging if the data of the blood oxygen and/or heart beat of the to-be-tested human is deviating from the corresponding safety data, rendering the to-be-tested human to be in a serious abnormal condition; and
a notification module 150, coupled to the processing module 130 for simultaneously notifying the related people and the medicare institution via the wireless network 170 or the mobile phone 160 by transmitting a notification signal when it is determined that it is in s serious abnormal condition due to deviation from the safety comparison,
whereby, the cloud life monitoring system is to timely monitor the data of the blood oxygen and/or heart beat of the to-be-tested human and to provide timely rescuing in emergency, so as to build a simple and efficient ICU system.

In the cloud life monitoring system 100 of the invention, the data of blood oxygen and/or heart of the to-be-tested human transferred by the mobile phone 160 is detected via a blood oxygen and/or heart beat detecting device (not shown in the drawing) and transferred to the mobile phone utilizing an APP with Bluetooth transferring function.

Further, in the cloud life monitoring system 100 of the invention, the wireless network 170 is a LINE (trademark) network or other public wireless network.

In addition, in the cloud life monitoring system 100 of the invention, the notification signal of the notification module is a voice message or alarm signal.

Moreover, in the cloud life monitoring system of the invention, the medicare institution includes medicare staff and medical facility.

What is claimed is:

1. A cloud life monitoring system for timely monitoring data of blood oxygen and/or heart beat of a to-be-tested human, comprising a cloud computer for executing software for cloud monitoring, including:
an input module for imputing data of blood oxygen and/or heart beat of the to-be-tested human timely, and continuously transferring the data from a mobile phone via a wireless network;
a processing module, coupled to the input module for receiving the data of the blood oxygen and/or heart beat and processing;
a storing module, coupled with the processing module, for storing safety data record of the blood oxygen and/or heart beat, historic record data of the blood oxygen and/or heart beat of the to-be-tested human, and the timely data of the blood oxygen and/or heart beat of the to-be-tested human, and for storing ID authentication data of related people and pre-set data of a medicare institution, in which the related people is the to-be-tested human or a people for taking care,
wherein the processing module further includes a comparison sub-module for comparing the timely data of the blood oxygen and/or heart beat of the to-be-tested human with its corresponding safety data, and for judging if the data of the blood oxygen and/or heart beat of the to-be-tested human is deviating from the corresponding safety data, rendering the to-be-tested human to be in a serious abnormal condition; and
a notification module, coupled to the processing module for simultaneously notifying the related people and the medicare institution via the wireless network or the mobile phone by transmitting a notification signal when it is determined that it is in a serious abnormal condition due to deviation from the safety data after comparison,
whereby, the cloud life monitoring system is to timely monitor the data of the blood oxygen and/or heart beat of the to-be-tested human and to provide timely rescuing in emergency, so as to build a simple and efficient ICU system.

2. A cloud life monitoring system of claim 1, wherein the data of blood oxygen and/or heart beat of the to-be-tested human transferred by the mobile phone is detected via a blood oxygen and/or heart beat detecting device and transferred to the mobile phone utilizing an APP with a bluetooth transferring function.

3. A cloud life monitoring system of claim 1, wherein the wireless network is a LINE (trademark) network or other public wireless network.

4. A cloud life monitoring system of claim 1, wherein the notification signal of the notification module is a voice message or alarm signal.

5. A cloud life monitoring system of claim 1 wherein the medicare institution includes medical staff and medical facility.
